(19) United States
(12) Patent Application Publication
(10) Pub. No.: US 2009/0318941 A1
(43) Pub. Date: Dec. 24, 2009

(54) Self-Expandable Endovascular Device For Aneurysm Occlusion

(75) Inventors: Ivan Sepetka, Los Altos, CA (US); Maria G. Aboytes, Palo Alto, CA (US); Hong Thu Doan, Milpitas, CA (US); Ricardo Aboytes, East Palo Alto, CA (US); Steven Hochberg, New York, NY (US); Peter Costantino, Armonk, NY (US); Craig F. Friedman, Westport, CT (US); Arindam Datta, Hillsborough, NJ (US)

Correspondence Address: KING & SPALDING, 1185 AVENUE OF THE AMERICAS, NEW YORK, NY, 10036-4003, US

(73) Assignee: BIOMERIX CORPORATION, New York, NY (US)

(21) Appl. No.: 12/294,210

(22) PCT Filed: Mar. 23, 2007
(86) PCT No.: PCT/US07/07320

§ 371 Date: Apr. 13, 2009

Related U.S. Application Data
(60) Provisional application No. 60/785,901, filed on Mar. 24, 2006.

(51) Int. Cl. A61B 17/08; A61M 25/00

(52) U.S. Cl. 606/158; 604/264

(57) ABSTRACT

The self-expandable endovascular apparatus for aneurysm occlusion of the invention comprises a deformable shape memory frame with at least a partial segment covering comprised of a matrix implant material. The device can be folded and/or stretched to adopt a narrow profile for loading into a coaxial delivery device and expands in place as it adopts its original shape on release from the device into an aneurysm. A method of treating an aneurysm, comprises the steps of: (a) providing the self-expandable endovascular apparatus inserted into a lumen of a delivery device comprising a proximal end and a distal end, the distal end having a distal tip; (b) advancing the distal tip of the delivery device into an opening in an aneurysm having an interior sac; (c) advancing the apparatus through the lumen into the opening; and (d) withdrawing the delivery device, whereby the apparatus expands into the sac and covers the opening.

### RELATED APPLICATIONS

This application incorporates by reference the entire specification of U.S. patent application Ser. No. 10/998,357 entitled “Aneurysm Treatment Devices and Methods” filed Nov. 26, 2004. The entire specifications of International Patent Application Numbers WO 2004/062531, published Jul. 29, 2004 and WO 2004/078023, published Sep. 16, 2004 are also herein incorporated by reference and are appended hereto as Exhibits 1 and 2.

### BACKGROUND

Current methods of treatment of aneurysms designed to fill the aneurysm lumen or sac by introducing medical devices, such as coils, often require deployment of multiple coils to seal the aneurysm and suffer from the problems associated with device compaction, such as recanalization of the aneurysm.

There is a need for a method of treatment of an aneurysm that provides a seal of the neck of the aneurysm that permits tissue regrowth leading to a permanent repair, and wherein the seal is not subject to recanalization and consequent reemergence of the aneurysm.

### SUMMARY OF THE INVENTION

The present invention provides an apparatus for aneurysm repair that includes a self-expandable frame and a physiologically compatible, resiliently compressible, elastomeric reticulated matrix.

Embodiments of the present invention provide systems and methods for treating aneurysms. One embodiment of a system according to the present invention includes an apparatus for aneurysm repair having a self-expandable frame and a physiologically compatible, resiliently compressible, elastomeric reticulated matrix and a delivery device. An embodiment of a method of treating an aneurysm according to the present invention, includes the steps of: (a) providing an apparatus for aneurysm repair that includes a self-expandable frame and a physiologically compatible, resiliently compressible, elastomeric reticulated matrix, inserted into a lumen of a delivery device; the delivery device having a proximal end and a distal end, the distal end having a distal tip; (b) advancing the distal tip of the delivery device into an opening in an aneurysm having an interior sac; (c) advancing the apparatus through the lumen into the opening; and (d) withdrawing the delivery device, whereby the apparatus expands into the sac and covers the opening.

In one embodiment, the method includes a step of sizing the aneurysm in order to provide or select an apparatus for aneurysm repair according to the present invention with the best fit to the aneurysm to be addressed. Sizing of the aneurysm includes assessing the size of the aneurysm sac and/or the size of the aneurysm opening to determine a suitable size and configuration of the retention member or members, and the size and geometry of the frame of the aneurysm repair apparatus to be used.

A suitable size of frame of the apparatus is a size, which when fully expanded, is slightly smaller in each dimension than the equivalent dimension of the aneurysm sac, and thus fits snuggly into the aneurysm sac. Because the neck of the aneursym is in general smaller than the diameter of the aneurysm sac, the frame of the apparatus is secured and resists expulsion from the aneurysm.

In addition, the size of the neck or opening of the can be determined to aid in selection of an appropriately sized elastomeric matrix to cover or block the aneurysm opening. In a particular embodiment, the elastomeric matrix of the apparatus substantially seals the opening of the aneurysm. In another embodiment, the elastomeric matrix of the apparatus completely closes the opening of the aneurysm.

The present invention, in one embodiment of another of its aspects, provides an apparatus for aneurysm repair, wherein the apparatus includes a self-expandable frame and a physiologically compatible, resiliently compressible, elastomeric reticulated matrix, wherein the apparatus radially and/or circumferentially conforms to the aneurysm, thereby facilitating sealing of the aneurysm.

In another embodiment of one of its aspects, the present invention further provides a method for treating an aneurysm having an aneurysm wall, with an apparatus comprising a body having a proximal cylindrical portion and a distal portion, wherein the apparatus comprises a self-expandable frame and a physiologically compatible, resiliently compressible, elastomeric reticulated matrix. The method comprises the steps of: (a) providing the apparatus inserted into the lumen of a delivery device; (b) advancing the distal tip of the delivery device into the aneurysm; (c) advancing the apparatus from the delivery device to the aneurysm; (d) positioning the apparatus in the aneurysm; and (e) permitting the frame to expand into a fully expanded shape, or to expand until limited by the aneurysm wall.

According to another embodiment of one of its aspects, the present invention also provides an apparatus for securing a medical implant directed to aneurysm repair, wherein the apparatus includes: a retention member coupled to the implant and adapted for positioning in an aneurysm in a vascular tissue, the retention member comprising an expandable radial component for retaining the implant in the aneurysm.

### BRIEF DESCRIPTION OF THE FIGURES

The following figures depict embodiments of the invention and are intended for illustration purposes only. The figures are not intended to be interpreted as limitations to the scope of the claimed invention.

FIG. 1 (A): Spherical shape memory frame (1) arranged as spokes attached at each end to a nut and with a thin layer of matrix implant material attached to the frame as an external jacket.

FIG. 2 (B): Spherical shape memory frame (2) as in (A), or metallic coils (3) with only a partial covering comprised of a spherical segment of matrix implant material (4).

FIG. 3 (C): Complex memory shape self-expandable spherical frame having an elliptical patch of matrix implant material (5), in an embodiment of the present invention. Radiopaque markers (6) are attached to the arms for detection during delivery and deployment.

FIG. 4: Coaxial delivery system with delivery guide wire (1), and external sheath (5) to provide support for internal sheath, having soft tip section with the lead-screw (2). Frame of Nitinol arms (10) with radial shape memory. Proximal nitinol nut/coil is screwed onto lead-screw (4) and distal nitinol nut/coil is screwed onto lead-screw (3). Matrix implant material (6) is attached to nitinol memory coil (8) and folded and/or stretched for delivery.

FIG. 5: Coaxial delivery system after delivery: Stretched Nitinol arms (10) of the frame with radial shape memory. Lead-screw section (7) of the internal delivery sheath. Nitinol memory coil (8), stretched during delivery and is relaxed after detachment. Proximal section (9) of the internal delivery sheath.

FIG. 6: Expanded spherical shape memory frame after delivery and release from coaxial delivery system. Nitinol shape memory frame arms (10) radially expanded according to its retained shape memory.

### DETAILED DESCRIPTION OF THE INVENTION

The self-expandable apparatus of the invention may be constructed from any physiologically compatible matrix, attached to a self-expandable frame for delivery into the lumen of an aneurysm. The matrix can be any physiologically compatible matrix, such as for instance and without limitation, the Biomerix matrix described in U.S. Ser. No. 10/998,357 filed Nov. 26, 2004. The self-expandable frame can be constructed of any self-expandable material, such as a metallic frame, constructed from for instance, Nitinol wire.

The physiologically compatible matrix can be attached to the self-expandable frame of the self-expandable apparatus of the invention by any suitable method well known to those of skill in the art. For instance, the matrix can be sutured to the frame with a biocompatible suture material. Alternatively, the matrix can be glued to the frame. In another embodiment, the matrix can be heat-bonded to the frame, where the frame has been pre-coated with a suitable heat-activated polymer or adhesive.

The self-expandable apparatus of the invention can be constructed to conform to different shapes and sizes to accommodate a range of aneurysm sizes and shapes, with the goal of achieving a fit conforming to the wall of the aneurysm. By blocking the aperture or neck of the aneurysm, the self-expandable apparatus can seal the lumen of the aneurysm and thereby isolate it from the vasculature.

Platinum bodies of a size necessary for detection can also be incorporated into or onto the self-expandable frame to provide radiopacity for ease of following deployment of the apparatus and to aid in accurate placement within a target aneurysm.

In a particular aspect, the aneurysm repair apparatus of the invention includes a self-expandable frame and a physiologically compatible, resiliently compressible, elastomeric reticulated matrix. In one embodiment, the elastomeric matrix is a suitable substrate for tissue regeneration. The resiliently compressible, elastomeric matrix can be biodurable. Alternatively, the resiliently compressible, elastomeric matrix can be resorbable. In a particular embodiment, the reticulated elastomeric matrix is configured to permit cellular ingrowth and proliferation into the elastomeric matrix. In another particular example of the elastomeric matrix of the invention, the elastomeric matrix is hydrophobic.

In another particular embodiment, the elastomeric matrix includes an elastomer polymer selected from the group consisting of polycarbonate polyurethanes, polyester polyurethanes, polyether polyurethanes, polysiloxane polyurethanes, polyurethanes with mixed soft segments, polycarbonates, polyesters, polyethers, polysiloxanes, polyurethanes. Alternatively, the elastomeric matrix can include a mixture of two or more of the above polymers.

In still another embodiment, the elastomeric matrix is reticulated and endoporously coated with a coating material that enhances cellular ingrowth and proliferation. In one example of the above embodiment, the coating material includes a coating, which can be a foamed coating, of a biodegradable material such as for instance, collagen, fibronectin, elastin, hyaluronic acid or a mixture of any of the foregoing biodegradable materials.

In a particular embodiment, the self-expandable aneurysm-sealing apparatus of the invention can be used alone as a single device to seal the neck of the aneurysm, or in combination with an embolic device, such as for instance, a matrix implant such as a Biomerix matrix, as described in U.S. Ser. No. 10/998,357 filed Nov. 26, 2004, and/or one or more embolic coils, to fill the lumen of the aneurysm. When used with other embolic devices, the self-expanding apparatus of the invention can be deployed first to seal the aneurysm neck, followed by delivery of embolic device, or devices to fill the interior aneurysm sac, and thereby stabilize the repair of the aneurysm. One or more embolic devices can be delivered by the same delivery micro-catheter used to deliver the aneurysm sealing apparatus. The embolic device or devices can be delivered by the same microcatheter through the threaded opening of the nut (described below) attached to the matrix of the apparatus of the present invention that substantially seals the opening at the neck of the aneurysm.

Insertion of one or more coils, or matrix implants into the lumen of the sealed aneurysm offers the advantage of providing a scaffold to support contiguous tissue growth inside the aneurysm sac. The self-expanding apparatus of the invention can also serve as a “neck protection” device, by expanding until confined by the aneurysm walls and extending beyond the aneurysm neck inside the aneurysm sac, preventing unwarranted migration of any filler (such as coils and/or matrix etc.) out of the aneurysm neck into the artery to which it is connected.

Without wishing to be bound by any particular theory, it is believed that occlusion or sealing of the aneurysm by the apparatus of the present invention occurs first as the ‘patch’ formed by the resiliently compressible, elastomeric reticulated matrix of the expanded apparatus acts as a mechanical barrier which reduces the flow of blood from the parent vessel into and out of the aneurysm sac. The reticulated matrix acts as a thrombotic patch and the stagnation of flow initiates the thrombotic response characterized by formation of a platlet-fibrin clot. This stage is followed by organization of the clot and finally, in the last stage of the healing response, resorption and resolution of the clot into fibrovascular tissue. In a particular embodiment, the apparatus of the invention for aneurysm repair includes a self-expandable frame and a physiologically compatible, resiliently compressible, elastomeric reticulated matrix, wherein the apparatus radially and/or circumferentially conforms to the aneurysm walls, thereby facilitating sealing of the aneurysm.

The self-expandable apparatus of the invention permits total reconstruction of the parental artery by delivering a patch of the physiologically compatible matrix across the neck of the aneurysm, thereby providing a tissue scaffold to promote endothelial growth. Sealing the opening or neck of the aneurysm results in permanent aneurysm occlusion and eliminates the risk of recanalization of the aneurysm sac. This approach also offers the advantage of one time repair or “single-shot occlusion” by deployment of a single, appropriately sized matrix cap held in position by the self-expanded frame to seal the aneurysm opening. As such, the self-expandable aneurysm-sealing apparatus of the invention has the potential to significantly reduce operating room time and device utilization, leading to significant economic advantages.

In a particular embodiment the invention provides a self-expandable apparatus for securing a medical implant directed to aneurysm repair, wherein the apparatus includes: a retention member coupled to the implant and adapted for positioning in an aneurysm in a vascular tissue, and wherein the retention member includes an expandable radial component for retaining the implant in the aneurysm. In a particular aspect, the retention member resists an expulsive force. In one example, the retention member of the self-expandable apparatus is integral to the implant. In another example, the radial component comprises two or more at least partially radial members.

In another particular embodiment the invention provides an implant, for use in treating a defect such as an aneurysm in a vascular tissue, that includes a material having a composition and structure adapted for application to the defect and for biointegration into the vascular tissue when applied to the defect. The application to the defect in the vascular tissue can be insertion into the defect. In one particular aspect, the structure includes a scaffold, which can be a reticulated structure. In one example, the reticulated structure is resiliently compressible. In one example, the resiliently compressible reticulated structure can include an elastomeric material. The elastomeric material can be a biodurable material, such as for instance, microporous ePTFE (expanded polytetrafluoroethylene). Alternatively, the elastomeric material can be a biosorbable material. The bioabsorbable materials for use as the elastomeric matrix material of the apparatus of the invention can be any bioabsorbable materials, such as for instance, but not limited to polyglycolic acid-polylactic acid (PGA/PLA) copolymers. Other suitable bioabsorbable materials can be solids, gels or water absorbing hydrogels with different bioresorption rates.

In another particular example of the implant of the invention, the implant includes a self-expanding retention member which when inserted into the defect, is of a size and dimensions to fit the defect. In other words, the retention member expands to meet the walls of the aneurysm sac and thereby at least partially resist expulsion from the defect. In one embodiment the retention member has a radial component. In a particular embodiment the structure of the implant of the invention comprises interconnected networks of voids and/or pores encouraging cellular ingrowth of vascular tissue.

FIG. 1 shows a spherical shape memory Nitinol frame (1), with a thin layer of implant material attached to the frame as a external jacket by surgical sutures to create a delicate self-expanding hollow structure. The jacketted Nitinol sphere can be folded or stretched and loaded into a flexible tube, to allow the delivery through a catheter or over a guide wire. Once delivered to targeted site such as aneurysm or blood vessel, the spherical structure re-expands and is detached using controlled delivery system.

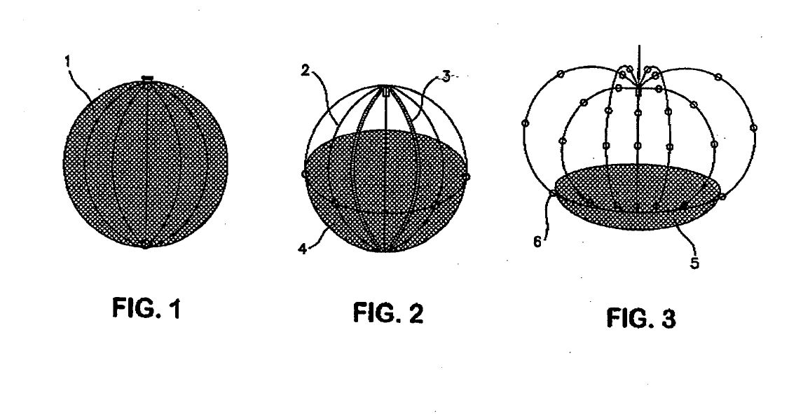

FIG. 2 illustrates an implant using the same expandable frame with a spherical segment of matrix implant material (4) attached to provide a lower profile for delivery. The self-expandable spherical frame is constructed using bare Nitinol wire arms (2), or Platinum coils (3). Platinum markers can also be added to provide the radiopacity of the implant structure during delivery and deployment. The Nitinol arms can be also constructed from different gauges of wires to provide different radial expansive force.

FIG. 3 Shows another design variation in which the complex memory shape self-expandable spherical structure has an elliptically shaped implant patch of matrix material. Complex memory shape can be used to provide optimal stability of the patch, especially in aneurysms with different sizes and shapes. Platinum markers attached to the arms can also be used to provide radiopacity during delivery and deployment. The elliptical segment of matrix material can be selected to fit and cover different anatomies of aneurysm neck presented by individual patients.

The self-expandable apparatus of the invention can be delivered to the aneurysm site using a controlled detachment system. In one aspect of an embodiment of the present invention, the controlled delivery and detachment system can be a coaxial delivery and detachment system.

The apparatus of the invention for aneurysm repair that includes a self-expandable frame and a physiologically compatible, resiliently compressible, elastomeric reticulated matrix can be folded and/or stretched on a guide-wire or on an internal sheath (that may harbor a guidewire), in order to attain a cross section narrow enough to be preloaded into a second sheath, the external sheath for use as a delivery catheter.

The physiologically compatible, resiliently compressible, elastomeric reticulated matrix can be of any thickness that retains sufficient flexibility to be folded and/or stretched to a collapsed form for loading onto a guidewire or inner sheath of a delivery microcatheter provided the collapsed apparatus has a sufficiently narrow profile to be threaded through the vasculature to the site of the aneurysm. In one embodiment, the thickness of the physiologically compatible, resiliently compressible, elastomeric reticulated matrix is in a range from about 100 um to about 1000 um (1 mm) when fully relaxed and expanded. In another embodiment, matrix is from about 200 um to about 800 um thick when fully relaxed and expanded. Alternatively, in a further embodiment, the matrix is from about 400 um to about 600 um (1 mm) thick when fully relaxed and expanded.

The porosity of the physiologically compatible, resiliently compressible, elastomeric reticulated matrix can be selected to permit cellular ingrowth. The average major dimension of the pores of the matrix can be optimized to encourage cellular ingrowth. In one embodiment, the pores have an average major dimension in a range from about 50 um to about 300 um. In another embodiment the pores have an average major dimension of from about 100 um to about 250 um. In still another embodiment the pores have an average major dimension of from about 150 um to about 200 um.

In a particular embodiment, the size of the delivery microcatheter ranges from about 0.018 inch to about 0.040 inch outside diameter (OD). For example, the OD of the delivery microcatheter can be 2 French (i.e. 0.026 inch/0.67mm) or 3 French (i.e. 0.039 inch/1.0 mm). In another particular embodiment, the inside diameter of the delivery microcatheter ranges from about 0.014 inch to about 0.021 inch).

The self-expandable apparatus of the invention can be designed to conform to a variety of sizes and shapes or geometries. The self-expandable aneurysm repair apparatus of the invention, when fully expanded, adopts a predetermined size and shape according to the shape memory of the metallic wire or other shape memory composition of the frame of the apparatus. In one embodiment, the apparatus when fully expanded can be any size from about 2 mm to about 20 mm, and can be any shape suited to fit a particular aneurysm sac. For instance and without limitation, the fully expanded apparatus can be spherical, elliptical, cylindrical or conical in shape.

In a particular embodiment, the self-expandable apparatus of the invention, when in its collapsed form, i.e when folded and/or stretched to be accommodated in a delivery microcatheter, has an OD of from about 2 French (i.e. 0.026 inch/0.67 mm) to about 5 French (i.e. 0.065 inch/1.7 mm). In one embodiment the collapsed apparatus, even when loaded into a microcather, maintains a high degree of flexibility so that the delivery device can be easily navigated through the vasculature. The collapsed apparatus can be loaded onto an internal sheath and the internal sheath carrying the collapsed apparatus can itself be loaded into an external sheath of a delivery catheter. Suitable external sheaths for delivery of the self-expanding apparatus of the invention can have an OD from about 3 French to about 6 French, or from about 6 French to about 7 French. The particular shape and dimensions of the self-expanding apparatus of the invention chosen to repair a particular aneurysm depend on the size of the aneurysm, which can be readily determined by the practitioner by standard tests and measurements using radiopaque dye to fill the aneurysm and aid in assessing its shape and dimensions. Aneurysms are generally from about 2 mm to about 20 mm in the largest dimension; small aneurysms can be from about 2 mm to about 4 mm; medium sized aneurysms are generally from about 5 mm to about 9 mm in the largest dimension; and the largest aneurysms are generally from about 10 mm to about 20 mm in the largest dimension; although even larger aneurysms are not unknown. Such “giant” aneurysms have been known to require up to 5 m of coils to fill.

In a particular embodiment of the invention, the size of the self-expanding apparatus of the invention chosen to repair a particular aneurysm is chosen to be slightly smaller than the size of the aneurysm. The longest dimension of the self-expanding apparatus is chosen to be slightly smaller than the longest dimension of the aneurysm and the shape of the apparatus is chosen to most nearly match the shape of the aneurysm.

In a one embodiment of the invention, the self-expanding apparatus of the invention can be from about 2 mm to about 20 mm in the longest dimension. In another embodiment, the self-expanding apparatus of the invention can be from about 4 mm to about 15 mm in the longest dimension. In still another embodiment, the self-expanding apparatus of the invention can be from about 5 mm to about 10 mm in the longest dimension. Alternatively, the self-expanding apparatus of the invention can be from about 6 mm to about 8 mm in the longest dimension. It is estimated that 80% of aneurysms are between about 3 mm and about 10 mm in the longest dimension.

Preferably, the delivery device is constructed to allow for optimal flexibility to navigate tortuous neuro-vasculature system. In one embodiment this is achieved with a guidewire of decreasing diameter from the proximal end (the end manipulated by the practitioner) to the distal end that delivers the self-expandable apparatus of the invention into the lumen of the aneurysm.

The present invention also provides a system for treating an aneurysm, the system includes a self-expandable apparatus constructed from a physiologically compatible matrix, attached to self-expandable frame for delivery into the lumen of an aneurysm, and a delivery device. The delivery device can be any suitable delivery device, such as for instance, a catheter or an endoscope-guided catheter, wherein the endoscope assists in navigation of the catheter to the site of deployment of the self-expandable apparatus of the invention for aneurysm repair.

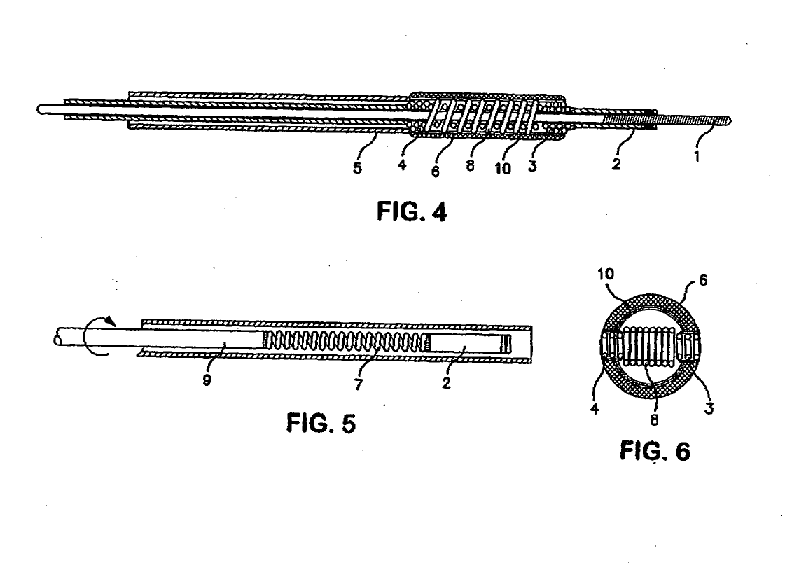

FIG. 4, shows a particular coaxial delivery system of the invention, constructed from a axial delivery guidewire (1), and an external delivery sheath (5) to provide support for internal sheath (9), having soft tip section (2) distally located to the fused lead-screw section (7). The soft tip section (2) is to navigate the system over the guide wire into the aneurysm or other targeted vasculature according to standard techniques for positioning a micro-catheter. The lead-screw (7) is to deliver and detach the implant having a nitinol memory coil (8). The foam matrix (6) is attached via the memory arms (10) to threaded nuts (3) and (4) as a jacket over the memory coil. Nuts(3) and (4) and memory coil (8) are step-wound as a single coil from the same strand of Nitinol wire. Nuts (3) and (4) have a smaller diameter and pitch adjusted to mesh with lead-screw (7) for delivery. Mid-coil (8) has a larger inside diameter to glide over the lead-screw when stretched during delivery, or when compressed during the detachment. In this example, between two to eight arms (10) with radial shape memory are welded to the nuts (3) and (4) to provide self-expansion capacity of the implant to the desired spherical or elliptical shape during the detachment from the delivery device and placement in the aneurysm lumen and seating of the self-expandable arms against the wall of the aneurysm sac.

The lead-screw (7) is first screwed onto proximal nut (4) all the way to the proximal end of the lead-screw, while stretching the implant memory coil and the arms into a straight position and engaging the distal screw until the distal tip of the lead-screw is screwed into distal nut 3. In this way the implant is locked in the stretched position and can be sheathed in external delivery sheath (5) for snaking through the vasculature to position the implant in the aneurysm and release into the aneurysm sac. A particular advantage of this system is the flexibility of the coil construction to provide good flexibility and tracking through the tortuous vascular system.

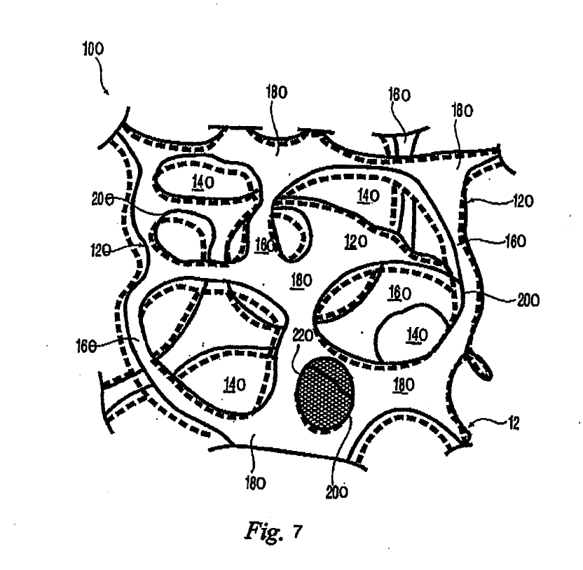

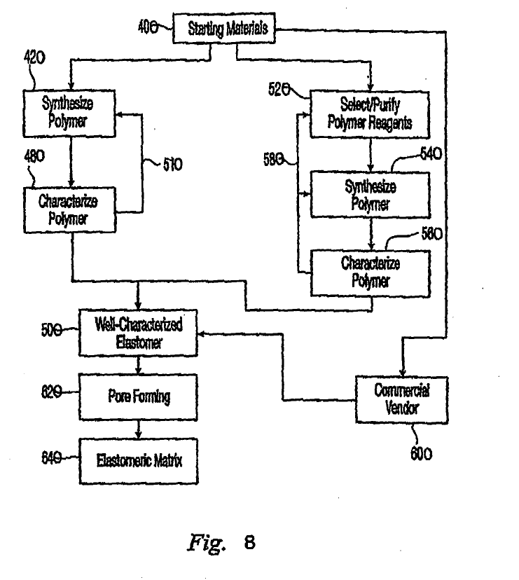

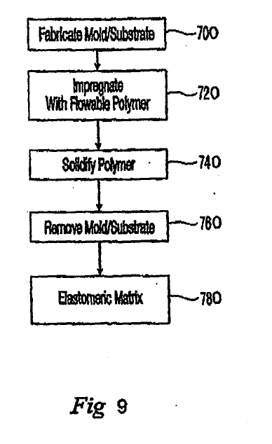

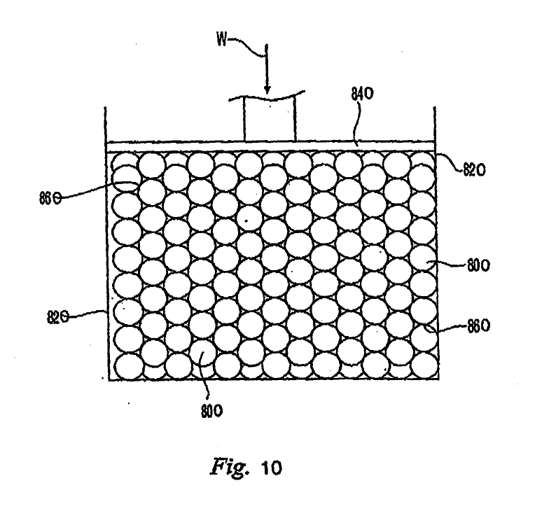

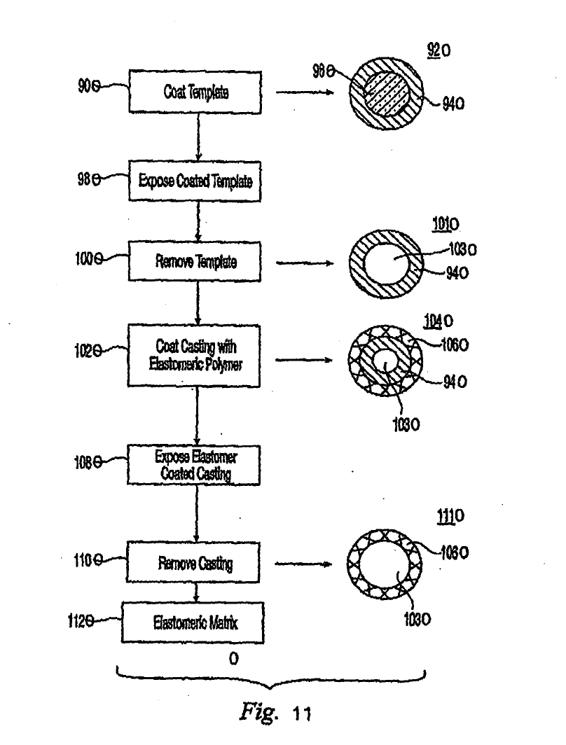

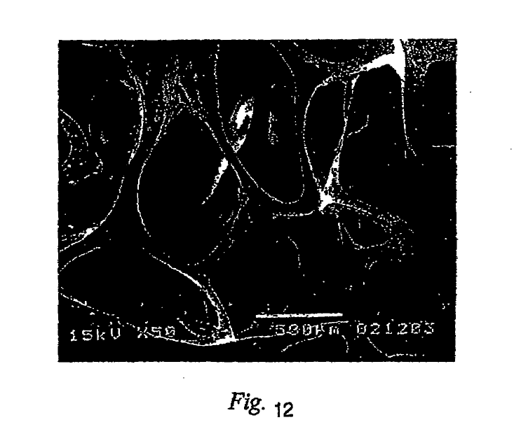

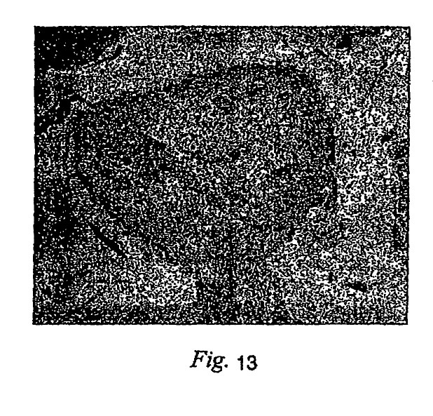

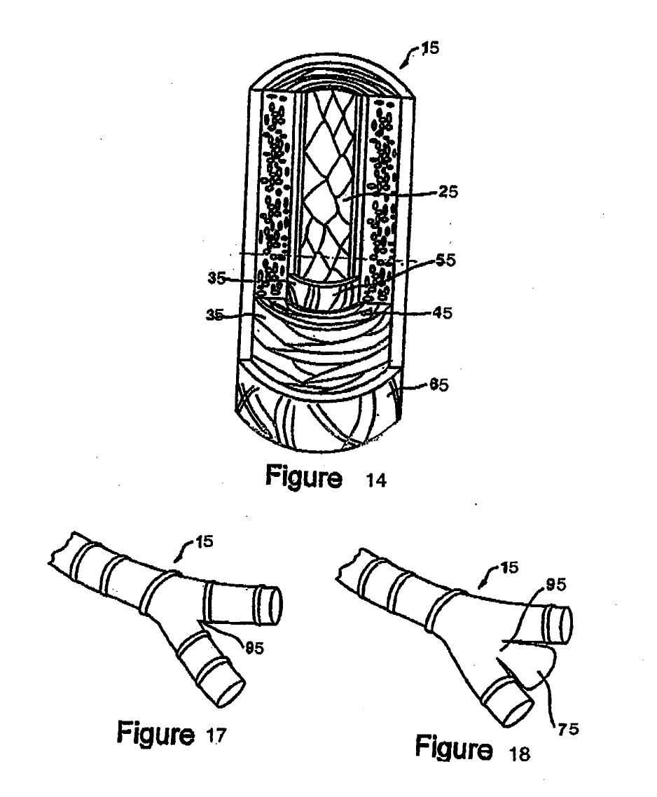

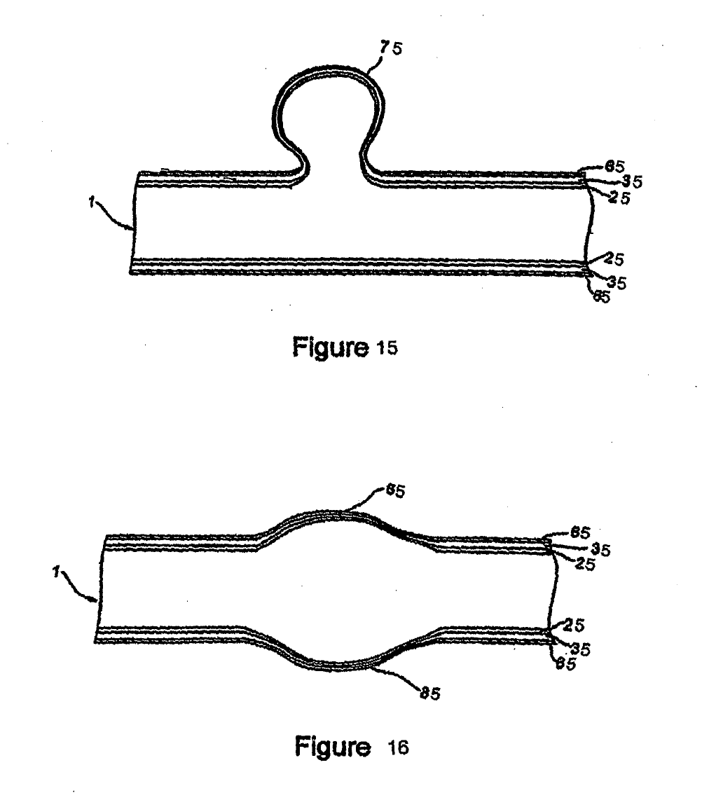

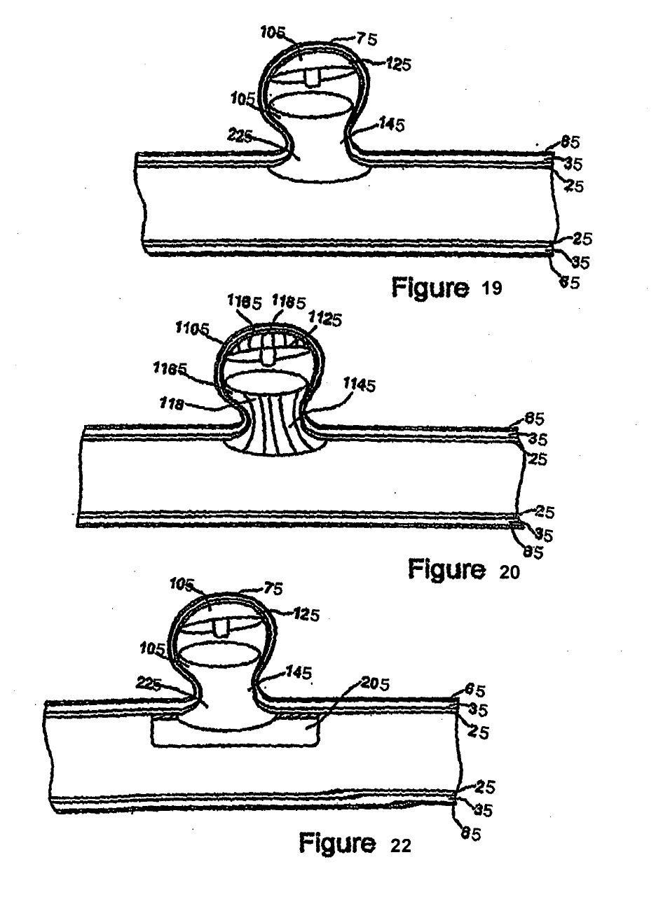

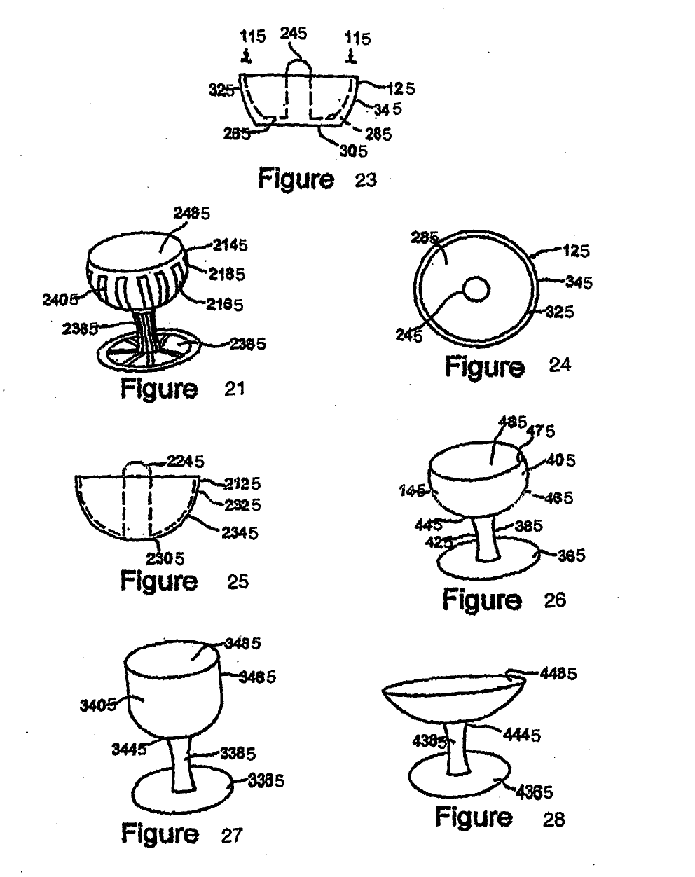

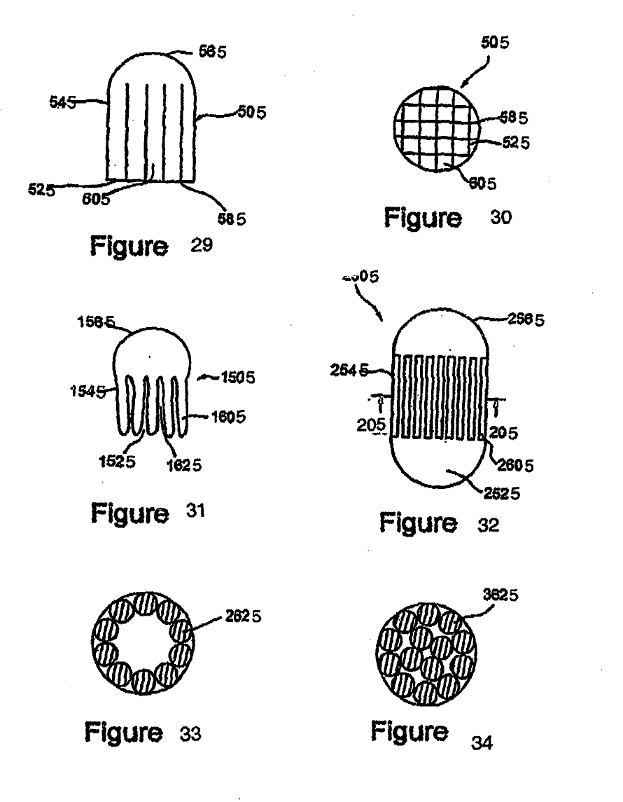

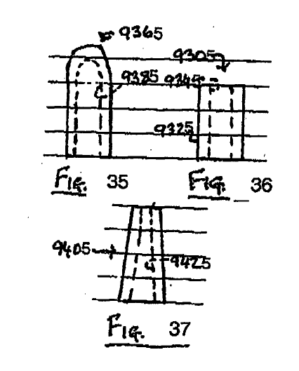

FIGS. 5 and 6 show an implant detached from the delivery device. External delivery sheath (5) is held still while torque is applied to internal sheath (9). The torque is transmitted to advance lead-screw (7) proximally and the memory coil begins to compress into it's retained memory shape. Pressure from arms (10) expands the implant into the desired spherical shape. The position of the implant can be adjusted to the optimal position and detached by unthreading and releasing from nut (3) and then from nut (4). Detachment occurs when the distal tip of the lead-screw (7) is un-screwed from the proximal nut (4). The distal tip of the internal sheath (2) cab then be pulled into external sheath (5) and the delivery device can be withdrawn.

The invention provides a high level of control during the detachment of the implant. In the event that the initial placement of the implant is not optimal, the partially expanded implant can be withdrawn back into the delivery device by reversing the process, i.e. by applying torque in the opposite direction to the direction of torque during the initial delivery attempt and collapsing the arms, rethreading the distal nut onto the distal tip of the lead-screw and withdrawing the implant back into the delivery device. Such non-optimal placement of the implant may occur for instance if the distal nut has been unthreaded and released from the distal tip of the lead-screw and the implant has partially expanded, but is either not accurately placed or has migrated into the parental artery from the initial delivery site. Withdrawal of the misplaced apparatus allows for subsequent redeployment and even permits multiple attempts to accurately position and fit the aneurysm-sealing apparatus to the desired location in difficult to reach aneurysms. The invention further provides a method of treating an aneurysm, wherein the method includes the steps of: (a) providing self-expandable apparatus constructed from a physiologically compatible matrix, attached to self-expandable frame for delivery into the lumen of an aneurysm, the apparatus being inserted into a lumen of a delivery device, the delivery device having a proximal end and a distal end, the distal end having a distal tip; (b) advancing the distal tip of the delivery device into an opening in an aneurysm having an interior sac; (c) advancing the apparatus through the lumen into the opening; and (d) withdrawing the delivery device, whereby the apparatus expands into the sac and covers the opening.

In a particular embodiment the delivery device of the invention is a catheter. In a particular aspect, the apparatus for aneurysm repair includes a radiopaque frame, or one or more radiopaque markers, or radiopaque retention members and deployment of the apparatus by the catheter can be assisted by visualization under fluoroscopy.

The invention also provides a method for treating an aneurysm having an aneurysm wall with an apparatus that includes a body having a proximal cylindrical portion and a distal portion, wherein the apparatus includes a self-expandable frame and a physiologically compatible, resiliently compressible, elastomeric reticulated matrix. The method includes the steps of: (a) providing the apparatus inserted into the lumen of a delivery device; (b) advancing the distal tip of the delivery device into the aneurysm; (c) advancing the apparatus from the delivery device to the aneurysm; (d) positioning the apparatus in the aneurysm; and (e) permitting the frame to expand into a fully expanded shape, or to expand until further expansion is limited by the aneurysm wall.

We claim:

1. An apparatus for aneurysm repair comprising a self-expandable frame and a physiologically compatible, resiliently compressible, elastomeric reticulated matrix.

2. The apparatus of claim 1, wherein the elastomeric matrix is a suitable substrate for tissue regeneration.

3. The apparatus of claim 1, wherein the resiliently compressible, elastomeric matrix is biodurable.

4. The apparatus of claim 1, wherein the resiliently compressible, elastomeric matrix is resorbable.

5. The apparatus of claim 2, wherein the reticulated elastomeric matrix is configured to permit cellular ingrowth and proliferation into the elastomeric matrix.

6. The apparatus of claim 5, wherein the reticulated elastomeric matrix is endoporously coated with a coating material that enhances cellular ingrowth and proliferation.

7. The apparatus of claim 6, wherein the coating material comprises a foamed coating of a biodegradable material, the biodegradable material comprising collagen, fibronectin, elastin, hyaluronic acid or mixtures thereof.

8. A system for treating an aneurysm, the system comprising an apparatus of claim 1 and a delivery device.

9. The system of claim 8, wherein the delivery device is a catheter.

10. A method of treating an aneurysm, comprising the steps of: (a) providing an apparatus of claim 1 inserted into a lumen of a delivery device comprising a proximal end and a distal end, the distal end having a distal tip; (b) advancing the distal tip of the delivery device into an opening in an aneurysm having an interior sac; (c) advancing the apparatus through the lumen into the opening; and (d) withdrawing the delivery device, whereby the apparatus expands into the sac and covers the aneurysm opening.

11. The method of claim 10, wherein the apparatus expands into the sac and substantially seals the aneurysm opening.

12. The method of claim 10, further comprising introducing one or more coil or embolic devices into the aneurysm sac and thereby to at least partially fill the aneurysm sac.

13. The method of claim 10, further comprising a step of assessing the size of the aneurysm.

14. The method of claim 10, further comprising a step of assessing the size of the opening of the aneurysm.

15. The method of claim 10, wherein the delivery device is a catheter.

16. An apparatus according to claim 1, wherein the apparatus radially and/or circumferentially conforms to the aneurysm, thereby facilitating sealing of the aneurysm.

17. A method for treating an aneurysm having an aneurysm wall with an apparatus comprising a body having a proximal cylindrical portion and a distal portion, wherein the apparatus comprises a self-expandable frame and a physiologically compatible, resiliently compressible, elastomeric reticulated matrix and the method comprises the steps of:
(a) providing the apparatus inserted into the lumen of a delivery device;
(b) advancing the distal tip of the delivery device into the aneurysm;
(c) advancing the apparatus from the delivery device to the aneurysm;
(d) positioning the apparatus in the aneurysm; and
(e) permitting the frame to expand into a fully expanded shape, or to expand until limited by the aneurysm wall.

18. The method according to claim 17, further comprising withdrawing the body of the apparatus at least partially back into the lumen of the delivery device, repositioning the apparatus relative to the aneurysm and repeating steps (c) through (e).

19. An apparatus for securing a medical implant directed to aneurysm repair, comprising: a retention member coupled to the implant and adapted for positioning in an aneurysm in a vascular tissue, the retention member comprising an expandable radial component for retaining the implant in the aneurysm.

20. The apparatus according to claim 19, further comprising a radiopaque marker.

21. The apparatus according to claim 19, wherein the retention member is integral to the implant.

22. The apparatus according to claim 19, wherein the radial component comprises two or more at least partially radial members.

23. The apparatus according to claim 19, wherein the retention member resists an expulsive force.

24. An implant for use in treating a defect in a vascular tissue, comprising a material having a composition and structure adapted for application to the defect and for biointegration into the vascular tissue when applied to the defect.

25. The implant according to claim 24, wherein the structure comprises a scaffold.

26. The implant according to claim 25, wherein the scaffold comprises a reticulated structure.

27. The implant according to claim 26, wherein the reticulated structure is resiliently compressible.

28. The implant according to claim 27, wherein the resiliently compressible reticulated structure comprises an elastomeric material.

29. The implant according to claim 28, wherein the elastomeric material comprises a biodurable material.

30. The implant according to claim 24, wherein application to the defect comprises insertion into the defect.

31. The implant according to claim 24, wherein the vascular defect is an aneurysm.

32. The implant according to claim 30, wherein the implant, when inserted into the defect, is dimensioned with respect to the defect to at least partially resist expulsion from the defect.

33. The implant according to claim 24, comprising a retention member having a radial component.

34. The implant according to claim 24, wherein the structure of the implant comprises interconnected networks of voids and/or pores encouraging cellular ingrowth of vascular tissue.

35. The apparatus of claim 1, wherein the elastomeric matrix is hydrophobic.

36. The apparatus of claim 1, wherein the elastomeric matrix comprises an elastomer selected from the group consisting of polycarbonate polyurethanes, polyester polyurethanes, polyether polyurethanes, polysiloxane polyurethanes, polyurethanes with mixed soft segments, polycarbonates, polyesters, polyethers, polysiloxanes, polyurethanes, and mixtures of two or more thereof.
